(19) United States
(12) Patent Application Publication
(10) Pub. No.: US 2024/0218446 A1
(43) Pub. Date: Jul. 4, 2024

(54) GENE MODIFICATION QUANTIFICATION IN SINGLE-CELL SEQUENCING

(71) Applicant: Mission Bio, Inc., South San Francisco, CA (US)

(72) Inventors: Shu Wang, South San Francisco, CA (US); Daniel Mendoza, South San Francisco, CA (US); Benjamin Schroeder, South San Francisco, CA (US)

(21) Appl. No.: 18/557,408

(22) PCT Filed: Apr. 27, 2022
(86) PCT No.: PCT/US2022/026578

§ 371 Date: Oct. 26, 2023

Related U.S. Application Data
(60) Provisional application No. 63/180,527, filed on Apr. 27, 2021.

(51) Int. Cl. C12Q 1/6881; C12Q 1/6869; G16B 30/00

(52) U.S. Cl. CPC C12Q 1/6881; C12Q 1/6869; G16B 30/00; C12Q 2600/156; C12Q 2600/16

(57) ABSTRACT

Methods described herein are useful for evaluating whether cells, that have previously undergone genetic modification, were successfully modified. Methods for determining whether a cell is a genetically modified cell involves a cell calling step and a dynamic normalization step that takes into consideration the variation of sequencing depth and number of working amplicons. Thus, the disclosed methods are able to more accurately and consistently identify successfully modified cells, even in situations where limited reads are available for analysis.

### CROSS REFERENCE TO RELATED APPLICATIONS

This application claims the benefit of and priority to U.S. Provisional Patent Application No. 63/180,527 filed Apr. 27, 2021, the entire disclosure of which is hereby incorporated by reference in its entirety for all purposes.

### BACKGROUND OF THE INVENTION

Cell and gene therapies aim to treat and prevent diseases, including cancer and inherited diseases, by altering the treatment landscape of intractable genetic disorders. For both ex-vivo and in-vivo cellular gene modification approaches, the biggest challenges are editing efficiency and precision. Successful clinical implementation of gene modification products requires precise assessment and reliable quality control at single-cell level. To date, there are no methods that achieve rapid, accurate, and reproducible readouts of genetically modified cells at the single-cell level.

### SUMMARY OF THE INVENTION

Methods disclosed herein include performing high-throughput single-cell DNA targeted sequencing, which enables comprehensive profiling of gene perturbation in thousands of cells. Additionally, methods disclosed herein provide several advantages over other currently used techniques which involve performing clonal outgrowth followed by qPCR to assess DNA integrations.

Disclosed herein is a method comprising: obtaining reads determined using a targeted sequencing panel, the reads sequenced from amplicons derived from a plurality of cells; calling one or more of the cells, wherein the calling comprises: identifying one or more cell barcodes in the obtained reads, wherein the one or more cell barcodes are identified at least for satisfying a total reads cutoff that is defined based on a number of amplicons of a first type in the targeted sequencing panel; attributing one of the identified one or more cell barcodes to one of the one or more cells; for each of one or more of the called cells, determining presence or absence of one or more amplicons of a second type comprising a barcode attributed to the called cell; determining whether the called cell is a genetically modified cell according to the presence or absence of one or more amplicons of the second type comprising the barcode attributed to the called cell.

In various embodiments, the plurality of cells are a plurality of human cells, and wherein the amplicons of the first type comprise human amplicons. In various embodiments, the total reads cutoff is not defined based on a number of amplicons of the second type in the targeted sequencing panel. In various embodiments, the amplicon of the second type is from a source that is foreign to the one or more cells. In various embodiments, the amplicon of the second type is a non-human amplicon. In various embodiments, the amplicon of the second type is a viral amplicon. In various embodiments, the viral amplicon is derived from a lentivirus.

In various embodiments, methods disclosed herein further comprise: after obtaining reads determined using a targeted sequencing panel, distinguishing between reads suspected to be sequenced from amplicons of the second type and reads sequenced from amplicons of the first type. In various embodiments, the total reads cutoff is defined as a product between the number of human amplicons in the targeted panel and a constant value X. In various embodiments, X is any one of 1, 2, 3, 4, 5, 6, 7, 8, 9, or 10. In various embodiments, X is 8.

In various embodiments, the one or more cell barcodes are each identified further based on a performance value of the targeted sequencing panel. In various embodiments, the performance value of the targeted sequencing panel is a product between a constant value Y and a mean coverage of a subset of reads. In various embodiments, the subset of reads represent reads comprising cell barcodes that satisfy the total reads cutoff. In various embodiments, Y is any one of 0.1, 0.2, 0.3, 0.4, 0.5, 0.6, 0.7, 0.8, 0.9, or 1. In various embodiments, Y is 0.2.

In various embodiments, the identified one or more cell barcodes in the obtained sequence reads represent functioning amplicons. In various embodiments, attributing one of the identified one or more cell barcodes to one of the one or more human cells comprises attributing each identified one or more cell barcodes to a corresponding human cell. In various embodiments, determining presence or absence of one or more amplicons of the second type comprises normalizing reads suspected to be sequenced from amplicons of the second type to reads sequenced from amplicons of the first type. In various embodiments, the normalization accounts for variations in sequencing depth and quantity of reads across different cells or across different samples. In various embodiments, determining presence or absence of one or more amplicons of the second type further comprises comparing the normalized reads suspected to be sequenced from amplicons of the second type to a percentage read cutoff. In various embodiments, the percentage read cutoff is between 0.05% and 0.5%. In various embodiments, the percentage read cutoff is any of 0.05%, 0.1%, 0.15%, 0.2%, 0.25%, 0.3%, 0.35%, 0.4%, 0.45%, or 0.5%. In various embodiments, determining whether the called cell is a genetically modified cell according to the presence or absence of one or more amplicons of the second type comprises determining that the called cell is a genetically modified cell when at least one amplicon of the second type is determined to be present. In various embodiments, determining whether the called cell is a genetically modified cell according to the presence or absence of one or more amplicons of the second type comprises determining that the called cell is a genetically modified cell when at least two, at least three, at least four, or at least five amplicons of the second type are determined to be present.

In various embodiments, genetically modified cells are successfully identified at a coefficient of variability (CV) percent of less than 1%. In various embodiments, genetically modified cells are successfully identified at a coefficient of variability (CV) percent of less than 0.9%, less than 0.8%, less than 0.7%, less than 0.6%, or less than 0.5%. In various embodiments, the method identifies a consistent mean percentage of genetically modified cells at 100% sampling and at 50% subsampling.

In various embodiments, the reads determined using a targeted sequencing panel are obtained by performing a single cell analysis. In various embodiments, the single cell analysis is one of a single cell DNA analysis or a single cell RNA analysis. In various embodiments, the targeted sequencing panel comprises at least 10 amplicons of the first type and at least 10 amplicons of the second type. In various embodiments, the targeted sequencing panel comprises at least 20 amplicons of the first type and at least 20 amplicons of the second type.

Additionally disclosed herein is a non-transitory computer readable medium comprising instructions that, when executed by a processor, cause the processor to: obtain reads determined using a targeted sequencing panel, the reads sequenced from amplicons derived from a plurality of cells; call one or more of the cells, wherein the instructions that cause the processor to call the one or more of the cells further comprises instructions that, when executed by the processor, cause the processor to: identify one or more cell barcodes in the obtained reads, wherein the one or more cell barcodes are identified at least for satisfying a total reads cutoff that is defined based on a number of amplicons of a first type in the targeted sequencing panel; attribute one of the identified one or more cell barcodes to one of the one or more cells; for each of one or more of the called cells, determine presence or absence of one or more amplicons of a second type comprising a barcode attributed to the called cell; determine whether the called cell is a genetically modified cell according to the presence or absence of one or more amplicons of the second type comprising the barcode attributed to the called cell. In various embodiments, the plurality of cells are a plurality of human cells, and wherein the amplicons of the first type comprise human amplicons. In various embodiments, the total reads cutoff is not defined based on a number of amplicons of the second type in the targeted sequencing panel. In various embodiments, the amplicon of the second type is from a source that is foreign to the one or more cells. In various embodiments, the amplicon of the second type is a non-human amplicon. In various embodiments, the amplicon of the second type is a viral amplicon. In various embodiments, the viral amplicon is derived from a lentivirus.

In various embodiments, non-transitory computer readable media disclosed herein further comprise instructions that, when executed by the processor, cause the processor to: after obtaining reads determined using a targeted sequencing panel, distinguish between reads suspected to be sequenced from amplicons of the second type and reads sequenced from amplicons of the first type. In various embodiments, the total reads cutoff is defined as a product between the number of human amplicons in the targeted panel and a constant value X. In various embodiments, X is any one of 1, 2, 3, 4, 5, 6, 7, 8, 9, or 10. In various embodiments, X is 8.

In various embodiments, the one or more cell barcodes are each identified further based on a performance value of the targeted sequencing panel. In various embodiments, the performance value of the targeted sequencing panel is a product between a constant value Y and a mean coverage of a subset of reads. In various embodiments, the subset of reads represent reads comprising cell barcodes that satisfy the total reads cutoff. In various embodiments, Y is any one of 0.1, 0.2, 0.3, 0.4, 0.5, 0.6, 0.7, 0.8, 0.9, or 1. In various embodiments, Y is 0.2. In various embodiments, the identified one or more cell barcodes in the obtained sequence reads represent functioning amplicons. In various embodiments, the instructions that cause the processor to attribute one of the identified one or more cell barcodes to one of the one or more human cells further comprises instructions that, when executed by the processor, cause the processor to attribute each identified one or more cell barcodes to a corresponding human cell. In various embodiments, the instructions that cause the processor to determine presence or absence of one or more amplicons of the second type further comprises instructions that, when executed by the processor, cause the processor to normalize reads suspected to be sequenced from amplicons of the second type to reads sequenced from amplicons of the first type.

In various embodiments, the normalization accounts for variations in sequencing depth and quantity of reads across different cells or across different samples. In various embodiments, the instructions that cause the processor to determine presence or absence of one or more amplicons of the second type further comprises instructions that, when executed by the processor, cause the processor to compare the normalized reads suspected to be sequenced from amplicons of the second type to a percentage read cutoff. In various embodiments, the percentage read cutoff is between 0.05% and 0.5%. In various embodiments, the percentage read cutoff is any of 0.05%, 0.1%, 0.15%, 0.2%, 0.25%, 0.3%, 0.35%, 0.4%, 0.45%, or 0.5%.

In various embodiments, the instructions that cause the processor to determine whether the called cell is a genetically modified cell according to the presence or absence of one or more amplicons of the second type further comprises instructions that, when executed by the processor, cause the processor to determine that the called cell is a genetically modified cell when at least one amplicon of the second type is determined to be present. In various embodiments, the instructions that cause the processor to determine whether the called cell is a genetically modified cell according to the presence or absence of one or more amplicons of the second type further comprises instructions that, when executed by the processor, cause the processor to determine that the called cell is a genetically modified cell when at least two, at least three, at least four, or at least five amplicons of the second type are determined to be present. In various embodiments, genetically modified cells are successfully identified at a coefficient of variability (CV) percent of less than 1%. In various embodiments, genetically modified cells are successfully identified at a coefficient of variability (CV) percent of less than 0.9%, less than 0.8%, less than 0.7%, less than 0.6%, or less than 0.5%. In various embodiments, the method identifies a consistent mean percentage of genetically modified cells at 100% sampling and at 50% subsampling. In various embodiments, the reads determined using a targeted sequencing panel are obtained by performing a single cell analysis.

In various embodiments, the single cell analysis is one of a single cell DNA analysis or a single cell RNA analysis. In various embodiments, the targeted sequencing panel comprises at least 10 amplicons of the first type and at least 10 amplicons of the second type. In various embodiments, the targeted sequencing panel comprises at least 20 amplicons of the first type and at least 20 amplicons of the second type.

### BRIEF DESCRIPTION OF THE SEVERAL VIEWS OF THE DRAWINGS

These and other features, aspects, and advantages of the present invention will become better understood with regard to the following description and accompanying drawings. It is noted that wherever practicable similar or like reference numbers may be used in the figures and may indicate similar or like functionality. For example, a letter after a reference numeral, such as “barcode 184A,” indicates that the text refers specifically to the element having that particular reference numeral. A reference numeral in the text without a following letter, such as “barcode 184,” refers to any or all of the elements in the figures bearing that reference numeral (e.g. “barcode 184” in the text refers to reference numerals “barcode 184A,” barcode 184B,” and/or “barcode 184C” in the figures).

FIG. 1A shows a single-cell processing of single cells to generate amplified nucleic acid molecules for sequencing, in accordance with an embodiment.

FIG. 1B shows a flow process of generating reads in the single-cell analysis, in accordance with an embodiment.

FIG. 2A is an example block diagram of a genetic modification analysis system, in accordance with an embodiment.

FIG. 2B is a flow diagram for identifying whether one or more cells underwent successful genetic modification, in accordance with an embodiment.

FIG. 3 depicts an example computing device for implementing system and methods described in reference to FIGS. 1A, 1B, 2A, and 2B.

FIG. 4A depicts a dynamic read normalization method for detecting genetically modified cells using example human (hg19) amplicons.

FIGS. 4B and 4C depict the observed percentages of modified cells across the five replicates from each concentration.

### DETAILED DESCRIPTION

### Definitions

Terms used in the claims and specification are defined as set forth below unless otherwise specified.

The term “subject” encompasses a cell, tissue, or organism, human or non-human, whether in vivo, ex vivo, or in vitro, male or female.

The term “sample” can include a single cell or multiple cells or fragments of cells or an aliquot of body fluid, such as a blood sample, taken from a subject, by means including venipuncture, excretion, ejaculation, massage, biopsy, needle aspirate, lavage sample, scraping, surgical incision, or intervention or other means known in the art.

The phrase “genetic modification” is used herein to refer to a process in which foreign DNA is introduced into one or more cells. In various embodiments, genetic modification of a cell involves introducing foreign DNA into a cell via viral or non-viral methods. For example, a viral vector or a virus (e.g., a lentivirus) can be used to introduce foreign DNA into a cell. The use of a viral mechanism for introducing foreign DNA into a cell is hereafter referred to as “genetic transduction.” In various embodiments, the process of genetic transduction involves the integration of the foreign DNA into the genome of the cell. In other embodiments, genetic transduction does not result in integration of the foreign DNA into the genome of the cell. Rather, the foreign DNA can remain within the cell. For example, genetic transduction can involve introduction of a foreign DNA plasmid that remains within the cell without being integrated into the genome of the cell. In various embodiments, genetic modification of a cell involves introducing foreign DNA into a cell via non-viral methods, an example of which includes using a non-viral vector (e.g., a polymer based vector, nanoparticles, or lipid vector). The use of a non-viral mechanism for introducing foreign DNA into a cell is hereafter referred to as “genetic transfection.”

The phrases “amplicons of a first type” and “amplicons of a second type” refer to amplicons that are derived from different sources. For example, amplicons of a first type may originate from a cell whereas amplicons of a second type may originate from a foreign source other than the cell. As used herein, although amplicons of a first type and amplicons of a second type may both be found in a cell, the amplicons of a second type may originate from a foreign source because foreign DNA has been introduced into the cell. In various embodiments, different sources refer to different organisms. For example, amplicons of a first type refers to human amplicons (e.g., amplicons from a human cell) and amplicons of second type refers to non-human amplicons (e.g., amplicons from a foreign source, such as viral amplicons derived from a virus). Furthermore, non-human amplicons can further include human genes that are introduced to a cell via a foreign source.

The term “analyte” refers to a component of a cell. Cell analytes can be informative for understanding a state, behavior, or trajectory of a cell. Therefore, performing single-cell analysis of one or more analytes of a cell using the systems and methods described herein are informative for determining a state or behavior of a cell. Examples of an analyte include a nucleic acid (e.g., RNA, DNA, cDNA), a protein, a peptide, an antibody, an antibody fragment, a polysaccharide, a sugar, a lipid, a small molecule, or combinations thereof. In particular embodiments, a single-cell analysis involves analyzing two different analytes such as protein and DNA. In particular embodiments, a single-cell analysis involves analyzing three or more different analytes of a cell, such as RNA, DNA, and protein.

A “barcode” nucleic acid identification sequence can be incorporated into a nucleic acid primer or linked to a primer to allow independent sequencing and identification to be associated with one another via a barcode which relates information and identification that originated from molecules that existed within the same sample. There are numerous techniques that can be used to attach barcodes to the nucleic acids within a discrete entity. For example, the target nucleic acids may or may not be first amplified and fragmented into shorter pieces. The molecules can be combined with discrete entities, e.g., droplets, containing the barcodes. The barcodes can then be attached to the molecules using, for example, splicing by overlap extension. In this approach, the initial target molecules can have “adaptor” sequences added, which are molecules of a known sequence to which primers can be synthesized. When combined with the barcodes, primers can be used that are complementary to the adaptor sequences and the barcode sequences, such that the product amplicons of both target nucleic acids and barcodes can anneal to one another and, via an extension reaction such as DNA polymerization, be extended onto one another, generating a double-stranded product including the target nucleic acids attached to the barcode sequence. Alternatively, the primers that amplify that target can themselves be barcoded so that, upon annealing and extending onto the target, the amplicon produced has the barcode sequence incorporated into it. This can be applied with a number of amplification strategies, including specific amplification with PCR or non-specific amplification with, for example, MDA. An alternative enzymatic reaction that can be used to attach barcodes to nucleic acids is ligation, including blunt or sticky end ligation. In this approach, the DNA barcodes are incubated with the nucleic acid targets and ligase enzyme, resulting in the ligation of the barcode to the targets. The ends of the nucleic acids can be modified as needed for ligation by a number of techniques, including by using adaptors introduced with ligase or fragments to allow greater control over the number of barcodes added to the end of the molecule.

It must be noted that, as used in the specification, the singular forms “a,” “an” and “the” include plural referents unless the context clearly dictates otherwise.

### Overview

Disclosed herein is a method for determining whether one or more cells are genetically modified cells. In various embodiments, a cell previously underwent genetic modification, and methods disclosed herein evaluate whether the cell underwent a successful genetic modification. Therefore, for cell populations, methods disclosed herein enable the identification of successfully genetically modified cells and/or determination of the efficiency of genetic modification across cells of the cell population. In particular embodiments, methods disclosed herein involve performing a single cell workflow process for processing individual cells to generate sequencing data. In particular embodiments, methods disclosed herein involve an in silico process that involves 1) a cell calling step for identifying cells and 2) a dynamic read normalization process for determining whether a called cell previously underwent successful genetic modification. In various embodiments, the dynamic read normalization method takes into consideration the variation of sequencing depth and number of working amplicons. Thus, by identifying a presence of foreign amplicons, called cells can be determined to have undergone a successful genetic modification. In particular embodiments, methods disclosed herein involve 1) genetic modification of a population of cells, 2) single cell workflow for processing individual cells of the population to generate sequencing data, and 3) an in silico process for calling cells and determining whether each cell underwent successful genetic modification. Altogether, methods disclosed herein are useful for evaluating the efficiency of the genetic modification process.

In various embodiments, different parties may perform the 1) genetic modification of a population of cells, 2) single cell workflow for processing individual cells of the population to generate sequencing data, and 3) an in silico process for calling cells and determining whether each cell underwent successful genetic modification.

In various embodiments, a first party performs the genetic modification of the population of cells and a second party performs the single cell workflow and in silico process. In such scenarios, the first party may genetically modify the population of cells and then provide the population of cells to the second party. Thus, the second party can perform the single cell and in silico analysis to evaluate the modification efficiency across the population of cells and provide the readout of the modification efficiency to the first party. In various embodiments, a first party performs the 1) genetic modification of a population of cells and further performs the 2) single cell workflow for processing individual cells of the population to generate sequencing data. A second party performs the in silico process for calling cells and determining whether each cell underwent successful genetic modification. In such a scenario, the first party may provide the sequencing data derived from the cells to the second party, such that the second party can perform the in silico analysis of the sequencing data. Thus, the second party can provide a readout of the modification efficiency to the first party. In various embodiments, a single party performs each of the 1) genetic modification of a population of cells, 2) single cell workflow for processing individual cells of the population to generate sequencing data, and 3) an in silico process for calling cells and determining whether each cell underwent successful genetic modification.

### Genetic Modification and Single Cell Analysis

In various embodiments, cells, such as a population of cells undergo a genetic modification, examples of which include a genetic transduction or genetic transfection. In various embodiments, the cells comprise human cells. The genetic modification may involve introducing foreign DNA from a foreign source to the human cells.

In various embodiments, the cells undergo genetic modification in bulk. A population of cells may be pooled together and exposed to a mechanism that introduces foreign DNA into the population of cells. For example, the population of cells can be present in a well, and foreign DNA may be introduced to the population of cells in the well. In various embodiments, genetic modification is performed on a cell population comprising at least 10 cells. In various embodiments, genetic modification is performed on a cell population comprising at least 10², 10³, 10⁴, 10⁵, 10⁶, 10⁷, 10⁸, or 10⁹ cells.

In various embodiments, foreign DNA is introduced via a viral mechanism, such as a viral vector or a virus (e.g., retrovirus, lentivirus, adenovirus, adeno-associated virus, and herpes virus). Further details of example viral mechanisms for introducing foreign DNA to a cell are described in Chong Z X, et al., Transfection types, methods and strategies: a technical review. PeerJ. 2021; 9:e11165, which is hereby incorporated by reference in its entirety. In various embodiments, foreign DNA is introduced via a non-viral mechanism, such as a non-viral vector (e.g., a polymer based vector, nanoparticles, or lipids). Further details of example non-viral vectors is described in Al-Dosari et al. Nonviral gene delivery: principle, limitations, and recent progress.” AAPS J. 2009; 11(4):671-681, which is incorporated by reference in its entirety.

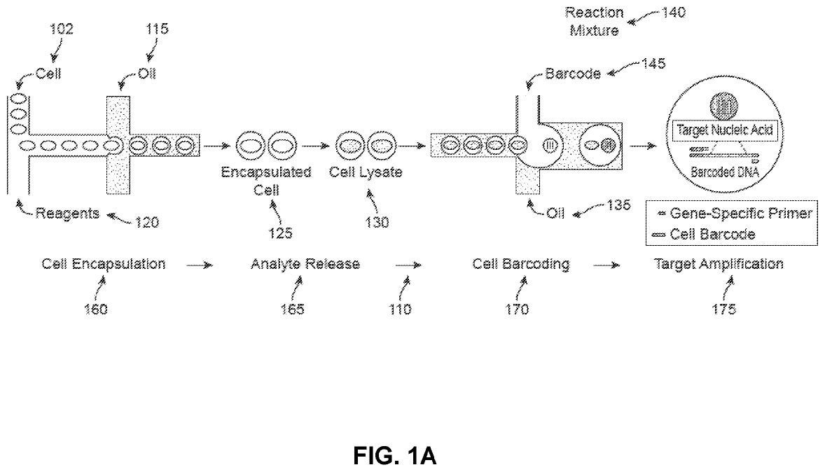

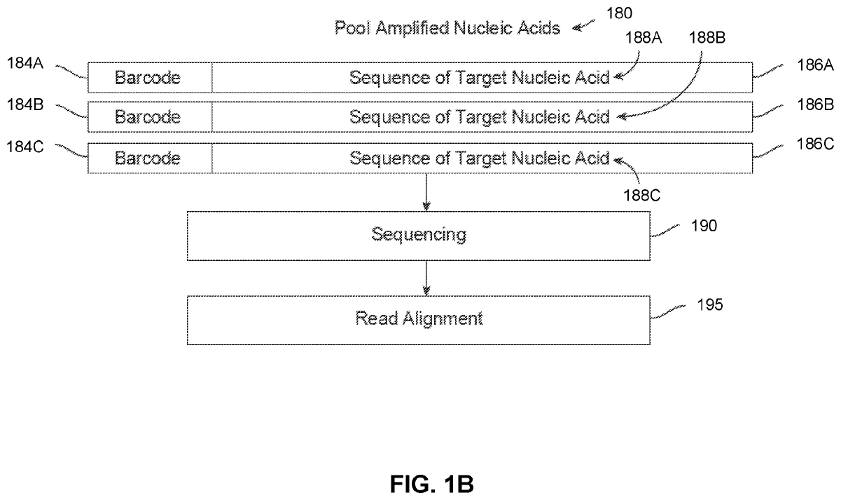

Reference is now made to FIG. 1A, which shows a single-cell processing of cells to generate amplified nucleic acid molecules for sequencing, in accordance with an embodiment. As shown in FIG. 1A, the single-cell workflow process may involve steps of cell encapsulation 160, analyte release 165 in droplets, cell barcoding 170 in droplets, and performing nucleic acid amplification in droplets. In various embodiments, the single-cell workflow implements a targeted panel (e.g., targeted DNA panel) for amplification of certain regions of interest. Further details of such a single-cell workflow is described in U.S. Pat. No. 10,161,007, which is hereby incorporated by reference in its entirety. In various embodiments, the flow pipeline shown in FIG. 1 is applicable for a Tapestri® workflow instrument.

As shown in FIG. 1A, single cells 102 are analyzed in the single cell workflow process beginning with the cell encapsulation 160 step. In various embodiments, the single cell workflow involves analyzing at least 10², at least 10³, at least 10⁴, at least 10⁵, at least 10⁶, at least 10⁷, at least 10⁸, or at least 10⁹ cells. As described herein, the cells 102 may have previously undergone a genetic modification. For example, the cells may have been exposed to foreign DNA such that one or more of the cells 102 includes the foreign DNA within the cell. In various embodiments, at least 10% of the cells 102 that are analyzed using the single cell workflow process shown in FIG. 1A include the foreign DNA within the cell. In various embodiments, at least 15%, at least 20%, at least 25%, at least 30%, at least 35%, at least 40%, at least 45%, at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7% at least 99.8%, or at least 99.9% of the cells 102 that are analyzed using the single cell workflow process shown in FIG. 1A include the foreign DNA within the cell. In various embodiments, 100% of the cells 102 that are analyzed using the single cell workflow process shown in FIG. 1A include the foreign DNA within the cell.

Generally, the cell encapsulation step 160 involves encapsulating a single cell 102 with reagents 120 into an emulsion. In various embodiments, the emulsion is formed by partitioning aqueous fluid containing the cell 102 and reagents 120 into a carrier fluid (e.g., oil 115), thereby resulting in an aqueous fluid-in-oil emulsion. The emulsion includes encapsulated cell 125 and the reagents 120. The encapsulated cell undergoes an analyte release at step 165. Generally, the reagents cause the cell to lyse, thereby generating a cell lysate 130 within the emulsion. In particular embodiments, the reagents 120 include proteases, such as proteinase K, for lysing the cell to generate a cell lysate 130. The cell lysate 130 includes the contents of the cell, which can include one or more different types of analytes (e.g., RNA transcripts, DNA, protein, lipids, or carbohydrates). In various embodiments, the different analytes of the cell lysate 130 can interact with reagents 120 within the emulsion. For example, primers in the reagents 120, such as reverse primers, can prime the analytes.

The cell barcoding step 170 involves encapsulating the cell lysate 130 into a second emulsion along with a barcode 145 and/or reaction mixture 140. In various embodiments, the second emulsion is formed by partitioning aqueous fluid containing the cell lysate 130 into immiscible oil 135. As shown in FIG. 1A, the reaction mixture 140 and barcode 145 can be introduced through a separate stream of aqueous fluid, thereby partitioning the reaction mixture 140 and barcode into the second emulsion along with the cell lysate 130.

Generally, a barcode 145 can label a target analyte to be analyzed (e.g., a target nucleic acid), which allows subsequent identification of the origin of a sequence read that is derived from the target nucleic acid. In various embodiments, multiple barcodes 145 can label multiple target nucleic acid of the cell lysate, thereby allowing the subsequent identification of the origin of large quantities of sequence reads. In various embodiments, barcodes 145 are attached to a bead. In various embodiments, the second emulsion has a single bead with barcodes facilitating subsequent identification any sequence read having the bead-specific barcode as originating from the emulsion.

In various embodiments, the target nucleic acid is a nucleic acid molecule of the cell 102. For example, the target analyte is a DNA or RNA molecule of the cell 102. In various embodiments, the target analyte is a foreign nucleic acid molecule e.g., a foreign nucleic acid molecule resulting from the genetic modification. For example, the target analyte may be a foreign DNA molecule that was introduced to the cell 102. As another example, the target analyte may be a foreign RNA molecule that was transcribed from a foreign DNA molecule introduced to the cell 102.

In various embodiments, a targeted sequencing panel is implemented for generating amplicons from the target nucleic acid. In various embodiments, the targeted sequencing panel is a targeted DNA sequencing panel for generating amplicons from target DNA molecules. In various embodiments, the targeted sequencing panel is a targeted RNA sequencing panel for generating amplicons from target RNA molecules. As described above, the target nucleic acid molecule may originate from the cell 102 or the target nucleic acid molecule may be a foreign nucleic acid molecule originating from a foreign source. In particular embodiments, a targeted sequencing panel can be implemented to generate amplicons from both target nucleic acid molecules originating from the cell 102 and foreign nucleic acid molecules originating from a foreign source. Primers of the targeted sequencing panel may be designed to hybridize with a sequence of a target nucleic acid molecule to enable priming and subsequent amplification. In various embodiments, the primers are linked to a barcode sequence. The subsequent amplification steps incorporate the barcode sequence into the amplicons, thereby enabling tracing of the amplicon back to a cell of origin based on presence of the barcode sequence.

The reaction mixture 140 allows the performance of a reaction, such as a nucleic acid amplification reaction. The target amplification step 175 involves amplifying target nucleic acids. For example, target nucleic acids of the cell lysate undergo amplification using the reaction mixture 140 in the second emulsion, thereby generating amplicons derived from the target nucleic acids. Although FIG. 1A depicts cell barcoding 170 and target amplification 175 as two separate steps, in various embodiments, the target nucleic acid is labeled with a barcode 145 through the nucleic acid amplification step.

As referred herein, the workflow process shown in FIG. 1A is a two-step workflow process in which analyte release 165 from the cell occurs separate from the steps of cell barcoding 170 and target amplification 175. For example, analyte release 165 from a cell occurs within a first emulsion followed by cell barcoding 170 and target amplification 175 in a second emulsion. In various embodiments, alternative workflow processes (e.g., workflow processes other than the two-step workflow process shown in FIG. 1A) can be employed. For example, the cell 102, reagents 120, reaction mixture 140, and barcode 145 can be encapsulated in an emulsion. Thus, analyte release 165 can occur within the emulsion, followed by cell barcoding 170 and target amplification 175 within the same emulsion.

Reference is now made to FIG. 1B, which shows a flow process of generating reads in the single-cell analysis, in accordance with an embodiment. Specifically, FIG. 1B depicts the steps of pooling amplified nucleic acids at step 180, sequencing the amplified nucleic acids at step 190, and performing read alignment at step 195. Generally, the flow process shown in FIG. 1B is a continuation of the workflow process shown in FIG. 1A.

For example, after target amplification at step 175 of FIG. 1A, the amplified nucleic acids 186A, 186B, and 186C are pooled at step 180 shown in FIG. 1A. For example, emulsions of amplified nucleic acids are pooled and collected, and the immiscible oil of the emulsions is removed. Thus, amplified nucleic acids from multiple cells can be pooled together. FIG. 1B depicts three amplified nucleic acids 186A, 186B, and 186C but in various embodiments, pooled nucleic acids can include hundreds, thousands, or millions of nucleic acids derived from analytes of multiple cells.

In various embodiments, each amplified nucleic acid 186 includes at least a sequence of a target nucleic acid 188 and a barcode 184. In various embodiments, an amplified nucleic acid 186 can include additional sequences, such as any of a universal primer sequence (e.g., an oligo-dT sequence), a random primer sequence, a gene specific primer forward sequence, a gene specific primer reverse sequence, or one or more constant regions (e.g., PCR handles).

In various embodiments, the amplified nucleic acids 186A, 186B, and 186C are derived from the same single cell and therefore, the barcodes 184A, 184B, and 184C are the same. As such, sequencing of the barcodes 184 allows the determination that the amplified nucleic acids 186A, 186B, and 186C are derived from the same cell. In various embodiments, the amplified nucleic acids 186A, 186B, and 186C are pooled and derived from different cells. Therefore, the barcodes 184A, 184B, and 184C are different from one another and sequencing of the barcodes 184 allows the determination that the amplified nucleic acids 186 are derived from different cells.

At step 190, the pooled amplified nucleic acids 186 undergo sequencing to generate sequence reads. For each amplified nucleic acid, the sequence read includes the sequence of the barcode and the target nucleic acid. Sequence reads originating from individual cells are clustered according to the barcode sequences included in the amplified nucleic acids. In various embodiments, at step 195, one or more sequence reads for each single cell are aligned (e.g., to a reference genome). Aligning the sequence reads to the reference genome allows the determination of where in the genome the sequence read is derived from.

In various embodiments, aligning the sequence reads allows the identification of reads that were sequenced from amplicons derived from a foreign source, and reads sequenced from amplicons that originate from the cell. As one example, assuming the cell is a human cell and the reference genome is a human reference genome, then reads that were sequenced from human amplicons would align with a sequence of the human reference genome. Alternatively, reads that were sequenced from an amplicon that originates from a foreign source may not align with a sequence of the human reference genome. As another example, a reference genome may comprise both known human and viral genomes. Thus, both reads from human amplicons and reads from viral amplicons can be aligned to the human and viral genomes of the reference genome.

In various embodiments, reads aligned to the reference genome can be distinguished based on one or more nucleotide differences between the reads and the reference genome. For example, a read that maps to the reference genome could be classified as viral in origin due to known nucleotide differences that exist between viral sequences and germline sequences.

### Genetic Modification Analysis System

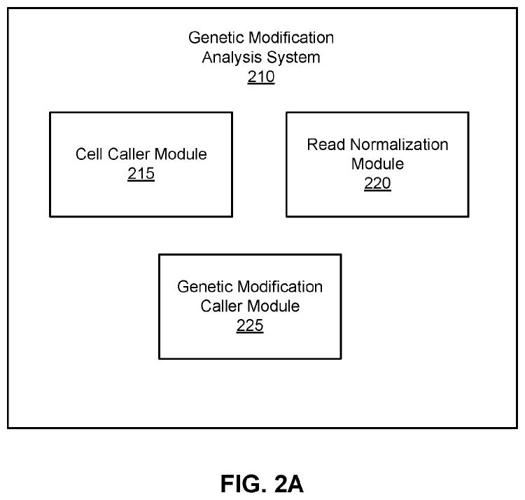

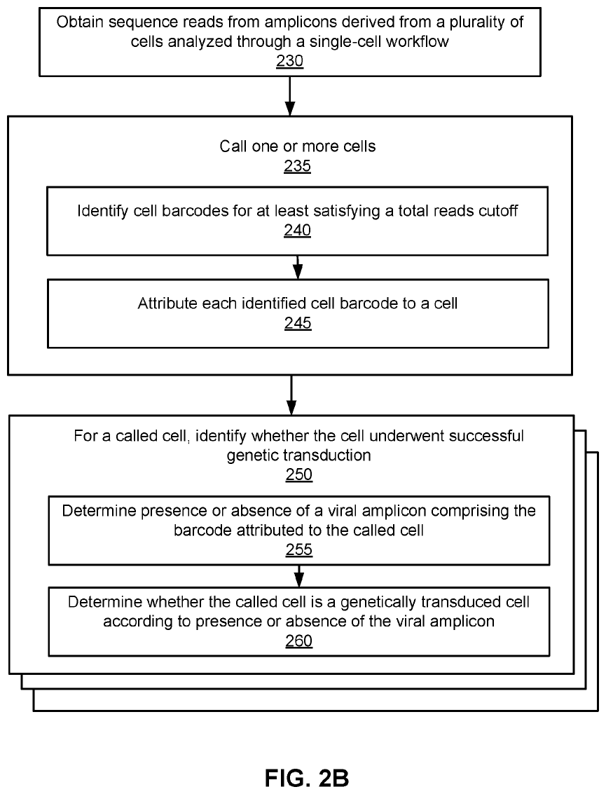

FIG. 2A is an example block diagram of a genetic modification analysis system 210, in accordance with an embodiment. Generally, the genetic modification analysis system 210 performs the in silico analysis that involves 1) a cell calling step for identifying cells and 2) a dynamic read normalization process for determining whether a called cell previously underwent successful genetic modification. The genetic modification analysis system 210 analyzes the sequencing data from the single-cell analysis (e.g., the sequence reads or aligned reads generated as a result of the process shown in FIG. 1). FIG. 2A introduces the individual modules of the genetic modification analysis system 210 including the cell caller module 215, the read normalization module 220, and the genetic modification caller module 225. Although FIG. 2A depicts the genetic modification analysis system 210 as including three separate modules, in various embodiments, additional or fewer modules may be included. Additionally, although the description below refers to particular functionality associated with each module, in various embodiments, the functionality may be differently arranged across the different modules.

Referring first to the cell caller module 215, it analyzes sequencing data obtained from the single-cell analysis and calls individual cells. Generally, the cell caller module 215 ensures that the sequencing data contains reads derived from amplicons that are indeed from a cell (and hence, the cell is called). In various embodiments, the cell caller module 215 calls cells by implementing one or more of the following steps: 1) identifying barcodes for cells that pass a total reads cutoff, 2) identifying functioning amplicons, and 3) identifying subset of the identified barcodes corresponding to functioning amplicons. In various embodiments, the cell caller module 215 calls cells by implementing each of the three steps of: 1) identifying barcodes for cells that pass a total reads cutoff, 2) identifying functioning amplicons, and 3) identifying subset of the identified barcodes corresponding to functioning amplicons. Altogether, the three steps, taken together, ensure that the cell caller module 215 is able to identify cells with higher accuracy.

The cell caller module 215 may categorize sequence reads according to the presence of barcode sequences in the reads. For example, the cell caller module 215 groups all sequence reads comprising a first barcode sequence in a first group, groups all sequence reads comprising a second barcode sequence in a second group, and so on. In various embodiments, the cell caller module 215 may generate tens, hundreds, thousands, or even millions of groups that is dependent on the total number of cells that were analyzed via the single-cell workflow.

Referring to the first step, for each barcode, the cell caller module 215 compares the number of reads comprising the barcode to a total reads cutoff. Here, the cell caller module 215 removes barcodes that appear in a number of reads below the total reads cutoff, because such barcodes may not be indicative of an actual cell, but may be a byproduct or artifact of the single-cell processing (e.g., partial genomic fragments from cell debris, cell-free DNA present in the sample, and background noise reads).

In various embodiments, the total reads cutoff is defined based on a number of amplicons of a first type included in the targeted sequencing panel. In particular embodiments, the amplicons of the first type refers to human amplicons (e.g., amplicons derived from the cell). In particular embodiments, the total reads cutoff is purposely not defined according to a number of amplicons of a second type included in the targeted sequencing panel. In various embodiments, the amplicons of the second type are amplicons originating from a foreign source different than a source of the amplicons of the first type. For example, the amplicons of the second type may be viral amplicons originating from a viral source. Here, amplicons of the second type may be present in the cells at inconsistent rates (e.g., due to poor signals and lack of consistent gene modification across the cells) and therefore, the purposeful exclusion of the amplicons of the second type from the total reads cutoff ensures more accurately identification of cells.

In various embodiments, the cell caller module 215 defines the total reads cutoff according to a number of amplicons of the first type (e.g., human amplicons). In various embodiments, the cell caller module 215 defines the total reads cutoff by the number of human amplicons in the panel times a constant value X. In various embodiments, X is any one of 1, 2, 3, 4, 5, 6, 7, 8, 9, or 10. In particular embodiments, the constant value X is 8. Therefore, the cell caller module 215 filters out and eliminates barcodes that show up in fewer reads than the total reads cutoff. The cell caller module 215 retains barcodes that show up in more reads than the total reads cutoff for subsequent analysis.

Referring next to the second step of identifying functioning amplicons, in various embodiments, the cell caller module 215 determines a performance value of the targeted sequencing panel and identifies amplicons that achieve a performance that is above the performance of the targeted sequencing panel. Thus, functioning amplicons are amplicons that are performing above a threshold value. Thus, by removing amplicons performing below the threshold value and only retaining those that exhibit performance above the performance of the targeted sequencing panel, the retained functioning amplicons are more likely to be derived from individual cells.

In various embodiments, the cell caller module 215 defines the performance of the targeted sequencing panel as a product between a constant value Y and a mean coverage of reads corresponding to barcodes that passed the total reads cutoff. In various embodiments, constant value Y is any one of 0.1, 0.2, 0.3, 0.4, 0.5, 0.6, 0.7, 0.8, 0.9, or 1. In particular embodiments, constant value Y is 0.2. Thus, such embodiments involve retaining the top 80% of the highest performing amplicons corresponding to barcodes that passed the total reads cutoff, while eliminating the bottom 20% of the lowest performing amplicons.

Reference is now made to the third step of identifying a subset of the identified barcodes corresponding to functioning amplicons. This analysis ensures that the subset of barcodes have sufficient read completeness. Specifically, the cell caller module 215 analyzes barcodes that passed the total reads cutoff to ensure that a sufficient number of functioning amplicons include the barcodes for read completeness. In various embodiments, the cell caller module 215 selects a subset of barcodes that passed the total reads cutoff for corresponding to at least Z % of functioning amplicons (e.g., functioning amplicons identified in the second step). In various embodiments, Z is any one of 10%, 20%, 30%, 40%, 50%, 60%, 70%, 80%, or 90%. In particular embodiments, Z % is 80%. To provide an example of this scenario, for a barcode that passed the total reads cutoff, the cell caller module 215 determines the total number of amplicons comprising that barcode. Then, the cell caller module 215 determines the total number of functioning amplicons (as determined in the prior analysis) comprising the barcode. If the total number of functioning amplicons comprising the barcode is greater than Z % of the total number of amplicons comprising the barcode, then the cell caller module 215 selects the barcode for inclusion in the subset. Conversely, if the total number of functioning amplicons comprising the barcode is less than Z % of the total number of amplicons comprising the barcode, then the cell caller module 215 excludes the barcode from the subset.

Having identified the subset of barcodes (e.g., barcodes that pass the total reads cutoff and correspond to at least Z % of functioning amplicons), the cell caller module 215 attributes barcodes of the subset to one or more cells. As discussed herein, each barcode is specific for a cell and its presence in an amplicon indicates that the amplicon is derived from the particular cell. Thus, in this step, the cell caller module 215 calls a cell for each barcode in the subset.

In various embodiments, the cell caller module 215 calls at least 100 cells, at least 200 cells, at least 300 cells, at least 400 cells, at least 500 cells, at least 600 cells, at least 700 cells, at least 800 cells, at least 900 cells, at least 1000 cells, at least 1500 cells, at least 2000 cells, at least 2500 cells, at least 3000 cells, at least 3500 cells, at least 4000 cells, at least 4500 cells, at least 5000 cells, at least 5500 cells, at least 6000 cells, at least 6500 cells, at least 7000 cells, at least 7500 cells, at least 8000 cells, at least 8500 cells, at least 9000 cells, at least 9500 cells, or at least 10000 cells.

Reference is now made to the read normalization module 220. Generally, the read normalization module 220 analyzes the called cells that were identified by the cell caller module 215, and determines whether each of the called cells has a presence or absence of amplicons of a second type (e.g., amplicons originating from a foreign source). Here, the read normalization module 220 implements a dynamic read normalization method that accounts for variations in sequencing depth and quantity of reads across different cells or across different samples. Altogether, the dynamic read normalization method enables more accurate identification of cells that have a presence or absence of amplicons of a second type (e.g., amplicons originating from a foreign source).

In various embodiments, the read normalization module 220 performs the dynamic read normalization method for one or more of the called cells. In various embodiments, the read normalization module 220 performs the dynamic read normalization method for each of the called cells. For each called cell, the read normalization module 220 identifies reads comprising a barcode of the called cell. From the reads comprising the barcode of the called cell, the read normalization module 220 normalizes reads that are suspected to be sequenced from amplicons of the second type (e.g., amplicons originating from a foreign source) to reads sequenced from amplicons of the first type (e.g., amplicons originating from the called cell). In various embodiments, the read normalization module 220 can distinguish reads sequenced from amplicons of a first type or a second type. For example, reads sequenced from amplicons of a first type (e.g., human amplicons) may have particular target sequences known to be present in a source of the first type. Reads sequenced from amplicons of a second type (e.g., foreign amplicons) may have particular target sequences known to be present in a source of the second type (e.g., a foreign source).

For example, assuming Amp_{foreign} are amplicons of the second type which represents the amplicons originating from the foreign source, and Amp_{cell} represents the amplicons of the first type which represents the amplicons originating from the cell, the read normalization module 220 may determine a normalized value of Amp_{foreign}/(Amp_{cell}+Amp_{foreign}). In the examples below, the normalized value may be further referenced as “hg19-nr % (hg19 normalized read %).” In various embodiments, the normalized value of Amp_{foreign}/(Amp_{cell}+Amp_{foreign}) may be less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, or less than 0.1%. Amp_{foreign} may, in some scenarios, be low in value due to poor or incomplete genetic modification, thereby leading to a low number of amplicons and a correspondingly low number of sequence reads. As described in further detail in the Examples below, low sequencing depth can be modeled at various subsampling levels. The dynamic read normalization method described herein enables accurate and reproducible detection of amplicons of the second type, even at low sequencing depth.

In various embodiments, the read normalization module 220 determines a presence or absence of one or more amplicons of the second type (e.g., foreign amplicons) by comparing the normalized value to a percentage read cutoff. In various embodiments, the percentage read cutoff is between 0.05% and 0.5%. In various embodiments, the percentage read cutoff is any of 0.05%, 0.1%, 0.15%, 0.2%, 0.25%, 0.3%, 0.35%, 0.4%, 0.45%, or 0.5%. In particular embodiments, the percentage read cutoff is 0.45%. In various embodiments, the percentage read cutoff value is selected because it represents a theoretical value that distinguishes between low performing amplicons of the second type and functioning amplicons of the second type. For example, assuming a targeted panel that generates 22 amplicons, in a theoretical, unbiased situation, each amplicon results in a read % of 4.5% (e.g., 100% of reads divided by the 22 amplicons). Here, the 4.5% read percent is amplified from 2 copies. For one copy, the estimated read % is 2.25%. For low performing amplicons (e.g., the lowest 20% amplicons), then the percentage is 2.25%*0.2, which yields a theoretical percentage read cutoff of 0.45%.

In various embodiments, if the normalized value of Amp_{foreign}/(Amp_{cell}+Amp_{foreign}) is above the percentage read cutoff, then the read normalization module 220 deems the amplicon of the second type to be present. In various embodiments, if the normalized value of Amp_{foreign}/(Amp_{cell}+Amp_{foreign}) is above the percentage read cutoff, then the read normalization module 220 deems the amplicon of the second type to be absent.

In various embodiments, the read normalization module 220 can repeat the process described above for each different amplicon of the second type that is included in the targeted panel. For example, assuming that there are “M” amplicons of the second type in the targeted panel. Thus, for each of the “M” amplicons of the second type in the targeted panel, the read normalization module 220 determines a normalized value for the amplicon, and compares the normalized value to determine whether the amplicon of the second type is present or absent. In one scenario, the read normalization module 220 may identify that 1 out of the “M” amplicons of the second type in the targeted panel is present, whereas the other “M−1” amplicons of the second type in the targeted panel are absent. In another scenario, the read normalization module 220 may identify that more than 1 amplicon of the second type is present. In yet another scenario, the read normalization module 220 may identify that zero amplicons of the second type are present.

Reference is now made to the genetic modification caller module 225. Generally, the genetic modification caller module 225 determines whether a called cell is a genetically modified cell according to the presence or absence of one or more amplicons of the second type. In various embodiments, if a single amplicon of the second type was determined to be present by the read normalization module 220, the genetic modification caller module 225 identifies the cell as a genetically modified cell. Returning to the example above, assume that the targeted panel includes “M” amplicons of the second type. If one or more of the “M” amplicons of the second type were determined to be present, then the genetic modification caller module 225 identifies the cell as genetically modified. If zero of the “M” amplicons of the second type were determined to be present, the genetic modification caller module 225 identifies the cell as non-genetically modified.

In various embodiments, the genetic modification caller module 225 determines that a called cell is a genetically modified cell if more than one amplicon of the second type was determined to be present. In various embodiments, the genetic modification caller module 225 determines that a called cell is a genetically modified cell if at least two, at least three, at least four, or at least five amplicons of the second type are determined to be present.

The genetic modification caller module 225 performs this analysis for each of the called cells. In various embodiments, the genetic modification caller module 225 identifies a total percentage of cells across the called cells that were identified as genetically modified cells. Here, this total percentage of cells identified as genetically modified cells can represent a readout as to the effectiveness or efficiency of the genetic modification. In various embodiments, the genetic modification caller module 225 identifies that at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7% at least 99.8%, or at least 99.9% of the cells are genetically modified cells.

In various embodiments, the genetic modification caller module 225 may determine the total percentage of cells that were identified as genetically modified cells across multiple runs. Thus, the consistency of the analysis across the multiple runs can be evaluated. In various embodiments, a coefficient of variation percentage (CV %) can be determined. Here, the CV % is a variability measure which represents the consistency of the analysis across multiple runs. CV % can be defined as: 100× sample standard deviation (STDEV.S)/mean. In various embodiments, across multiple runs, the genetic modification caller module 225 may determine a CV % of less than 1%. In various embodiments, across multiple runs, the genetic modification caller module 225 may determine a CV % of less than 0.9%, less than 0.8%, less than 0.7%, less than 0.6%, or less than 0.5%.

### Example Method for Identifying Whether a Cell is Genetically Modified

FIG. 2B is a flow diagram for identifying whether one or more cells underwent successful genetic modification, in accordance with an embodiment. Generally, as shown in FIG. 2B, the method includes two main steps, including a first step of calling the one or more cells (e.g., step 235) followed by a second step of identifying whether one or more of the called cells underwent successful genetic modification (e.g., step 250). The first step of calling the one or more cells considers a first type of amplicon (e.g., a human amplicon) and excludes a second type of amplicon (e.g., a viral amplicon), thereby enabling more accurate identification and calling of individual cells. Furthermore, the second step of identifying whether a cell underwent successful genetic modification involves the implementation of a dynamic normalization process that takes into consideration to cell to cell variability (e.g., variability arising in sequencing depth and/or quantity of amplicons). Thus, the process shown in FIG. 2B can identify whether cells have underwent successful genetic modification with improved accuracy and reliability (e.g., as is evidence by a lower coefficient of variation, as described in the Examples below).

Specifically, step 230 involves obtaining sequence reads from amplicons derived from a plurality of cells analyzed through a single cell workflow. For example, as is described in further detail herein, single cells may be encapsulated and lysed, followed by barcoding (e.g., using barcode sequences that identify the cell of origin) and amplification using a targeted panel. Thus, the single cell workflow can generate sequencing data for a plurality of cells that have undergone single cell analysis. Here, the sequencing data includes sequence reads from amplicons derived from the plurality of cells. In various embodiments, the sequencing data comprises sequence reads derived from RNA molecules (e.g., RNA sequencing data). For example, a targeted RNA panel may have been designed to target and amplify RNA molecules of interest. In various embodiments, the sequencing data comprises sequence reads derived from DNA molecules (e.g., DNA sequencing data). For example, a targeted DNA panel may have been designed to target and amplify DNA molecules of interest.

Step 235 involves calling one or more cells. As shown in FIG. 2B, step 235 may involve both step 240 and step 245. Step 240 involves identifying cell barcodes that at least satisfy a total reads cutoff. Thus, cell barcodes that fail to satisfy this threshold are eliminated. The total reads cutoff is dependent on a number of amplicons of a first type that are included in the targeted panel. For example, the amplicons of the first type are human amplicons. In various embodiments, the total reads cutoff is not dependent on a number of amplicons of a second type that are included in the targeted panel. For example, the amplicons of the second type are viral amplicons.

In various embodiments, having identified cell barcodes that satisfy the total reads cutoff, step 240 further involves further identifying cell barcodes that perform above a performance value of the targeted panel. This ensures that only high-performing cell barcodes are retained for subsequent analysis. For example, the performance value of the targeted panel may be represented by the product between a constant value Y and a mean coverage of reads that include cell barcodes that satisfy the total reads cutoff). In particular embodiments, Y is a value of 0.2.

Step 245 involves attributing each identified cell barcode (e.g., each cell barcode that satisfies the total reads cutoff) to a cell. For example, during the barcoding step in the single cell workflow, each cell barcode is introduced into a droplet and incorporated into nucleic acids and/or amplicons derived from a cell or cell lysate present in the droplet. Thus, presence of a cell barcode in an amplicon allows for the identification of the cell of origin.

Step 250 involves identifying whether a called cell underwent successful genetic modification. In various embodiments, step 250 can be performed multiple times for different called cells. For example, for each called cell, step 250 can be performed to determine whether each called cell underwent successful genetic modification. As shown in FIG. 2B, step 250 involves steps 255 and 260. Step 255 involves determining presence or absence of a viral amplicon comprising the barcode attributed to the called cell. Here, step 255 involves a dynamic normalization step. For example, for all reads that include the barcode attributed to the called cell, the reads are divided into two groups A first group of reads are suspected to have been sequenced from viral amplicons. A second group of reads are sequenced from human amplicons. The dynamic normalization includes normalizing the reads of the first group (e.g., reads suspected to have been sequenced from viral amplicons) to reads of the second group (e.g., reads sequenced from human amplicons). Here, the dynamic normalization step accounts for variations in sequencing depth and quantity of reads across different cells or across different samples. Step 255 further involves comparing a quantity of the normalized reads suspected to be sequenced from viral amplicons (e.g., also referred to as “normalized viral amplicon reads”) to a percentage read cutoff. In various embodiments, the percentage read cutoff is between 0.05% and 0.5%. Thus, if a quantity of normalized viral amplicon reads for the called cell is above the percentage read cutoff, a viral amplicon is deemed to be present. Conversely, if a quantity of normalized viral amplicon reads for the called cell is below the percentage read cutoff, a viral amplicon is deemed to be absent.

Step 260 involves determining whether the called cell is a genetically modified cell according to the presence or absence of one or more viral amplicons. For example, if at least one viral amplicon is present, then the called cell is deemed as a genetically modified cell. As another example, if zero viral amplicons are present, then the called cell is deemed to be a non-genetically modified cell.

In various embodiments, step 250 is performed multiple times for multiple called cells to determine whether each of the called cells underwent successful genetic modification. Although not shown in FIG. 2B, a subsequent step after 250 can involve determining a total percentage of cells that underwent successful genetic modification. In other words, a proportion of cells in a population of cells can be determined to have undergone successful genetic modification. This is a valuable readout for understanding the success of particular genetic modification methodologies.

### Targeted Panels

Embodiments disclosed herein include targeted panels for interrogating one or more target nucleic acids. Generally, the designing of a targeted panel involves designing and generating probes (e.g., primers) that target sequences of the target nucleic acids. For example, probes are designed and generated such that the probes can hybridize with target sequences of the target nucleic acids. Thus, in subsequent amplification steps, the probes are used to amplify the target sequences of the target nucleic acids to generate amplicons.

In particular embodiments, a target nucleic acid originates from the cell that is undergoing single cell analysis. For example, the target nucleic acid may correspond to a gene and therefore, the target sequencing panel is implemented to interrogate the target nucleic acid of the gene. In particular embodiments, a target nucleic acid originates from a foreign source other than the cell that is undergoing single cell analysis. For example, the target nucleic acid may be a foreign nucleic acid that was introduced into the cell. Therefore, the target sequencing panel is implemented to interrogate for the presence or absence of the foreign nucleic acid molecule in the cell.

In various embodiments, the targeted panel is designed to generate 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 125, 150, 175, 200, 250, 300, 350, 400, 450, 500, or 1000 different amplicons corresponding to different target nucleic acids. In various embodiments, the targeted panel is designed to generate between 10 and 100 different amplicons corresponding to different target nucleic acids. In various embodiments, the targeted panel is designed to generate between 20 and 80, between 25 and 70, between 30 and 60, between 35, and 50, or between 40 and 45 different amplicons corresponding to different target nucleic acids. In particular embodiments, the targeted panel is designed to generate between 42 and 45 different amplicons corresponding to different target nucleic acids. In particular embodiments, the targeted panel is designed to generate 44 different amplicons corresponding to different target nucleic acids.

In various embodiments, the targeted panel is designed to generate a first set of amplicons derived from a cell that is undergoing single cell analysis as well as a second set of amplicons derived from a foreign source other than the cell that is undergoing single cell analysis. In various embodiments, the targeted panel generates more amplicons of the first set (e.g., amplicons derived from a cell that is undergoing single cell analysis) in comparison to a number of amplicons for the second set (e.g., amplicons derived from a foreign source other than the cell that is undergoing single cell analysis). In various embodiments, the targeted panel generates less amplicons of the first set (e.g., amplicons derived from a cell that is undergoing single cell analysis) in comparison to a number of amplicons for the second set (e.g., amplicons derived from a foreign source other than the cell that is undergoing single cell analysis). In various embodiments, the targeted panel generates an equal number of amplicons of the first set (e.g., amplicons derived from a cell that is undergoing single cell analysis) and amplicons of the second set (e.g., amplicons derived from a foreign source other than the cell that is undergoing single cell analysis). In various embodiments, the targeted panel generates at least 20 amplicons of the first set (e.g., amplicons derived from a cell that is undergoing single cell analysis) and at least 20 amplicons of the second set (e.g., amplicons derived from a foreign source other than the cell that is undergoing single cell analysis). For example, the targeted panel may generate at least 20 human amplicons and at least 10 foreign amplicons from a foreign source. For example, the targeted panel may generate at least 20 human amplicons and at least 20 foreign amplicons from a foreign source. In particular embodiments, the targeted panel generates 22 amplicons of the first set (e.g., amplicons derived from a cell that is undergoing single cell analysis) and 15 amplicons of the second set (e.g., amplicons derived from a foreign source other than the cell that is undergoing single cell analysis). In particular embodiments, the targeted panel generates 22 amplicons of the first set (e.g., amplicons derived from a cell that is undergoing single cell analysis) and 16 amplicons of the second set (e.g., amplicons derived from a foreign source other than the cell that is undergoing single cell analysis). In particular embodiments, the targeted panel generates 22 amplicons of the first set (e.g., amplicons derived from a cell that is undergoing single cell analysis) and 22 amplicons of the second set (e.g., amplicons derived from a foreign source other than the cell that is undergoing single cell analysis).

### Barcodes and Barcoded Beads

Embodiments of the invention involve providing one or more barcode sequences for labeling analytes of a single cell during step 170 shown in FIG. 1A. The one or more barcode sequences are encapsulated in an emulsion with a cell lysate derived from a single cell. Barcode sequences are therefore cell specific. As such, the one or more barcodes label analytes of the cell, thereby allowing the subsequent determination that sequence reads derived from the analytes originated from the same single cell.

In various embodiments, a plurality of barcodes are added to an emulsion with a cell lysate. In various embodiments, the plurality of barcodes added to an emulsion includes at least 10², at least 10³, at least 10⁴, at least 10⁵, at least 10⁵, at least 10⁶, at least 10⁷, or at least 10⁸ barcodes. In various embodiments, the plurality of barcodes added to an emulsion have the same barcode sequence. For example, multiple copies of the same barcode label are added to an emulsion to label multiple analytes derived from the cell lysate, thereby allowing identification of the cell from which an analyte originates from. In various embodiments, the plurality of barcodes added to an emulsion comprise a ‘unique identification sequence’ (UMI). A UMI is a nucleic acid having a sequence which can be used to identify and/or distinguish one or more first molecules to which the UMI is conjugated from one or more distinct second molecules to which a distinct UMI, having a different sequence, is conjugated. UMIs are typically short, e.g., about 5 to 20 bases in length, and may be conjugated to one or more target molecules of interest or amplification products thereof. UMIs may be single or double stranded. In some embodiments, both a barcode sequence and a UMI are incorporated into a barcode. Generally, a UMI is used to distinguish between molecules of a similar type within a population or group, whereas a barcode sequence is used to distinguish between populations or groups of molecules that are derived from different cells. In some embodiments, where both a UMI and a barcode sequence are utilized, the UMI is shorter in sequence length than the barcode sequence. The use of barcodes is further described in US Patent Application Pub. No. US20180216160A1, which is hereby incorporated by reference in its entirety.

In some embodiments, the barcodes are single-stranded barcodes. Single-stranded barcodes can be generated using a number of techniques. For example, they can be generated by obtaining a plurality of DNA barcode molecules in which the sequences of the different molecules are at least partially different. These molecules can then be amplified so as to produce single stranded copies using, for instance, asymmetric PCR. Alternatively, the barcode molecules can be circularized and then subjected to rolling circle amplification. This will yield a product molecule in which the original DNA barcoded is concatenated numerous times as a single long molecule.

In some embodiments, circular barcode DNA containing a barcode sequence flanked by any number of constant sequences can be obtained by circularizing linear DNA. Primers that anneal to any constant sequence can initiate rolling circle amplification by the use of a strand displacing polymerase (such as Phi29 polymerase), generating long linear concatemers of barcode DNA.

In various embodiments, barcodes can be linked to a primer sequence that allows the barcode to label a target nucleic acid. In one embodiment, the barcode is linked to a forward primer sequence. In various embodiments, the forward primer sequence is a gene specific primer that hybridizes with a forward target of a nucleic acid. In various embodiments, the forward primer sequence is a constant region, such as a universal primer, that hybridizes with a complementary sequence attached to a gene specific primer. The complementary sequence attached to a gene specific primer can be provided in the reaction mixture (e.g., reaction mixture 140 in FIG. 1A). Including a constant forward primer sequence on barcodes may be preferable as the barcodes can have the same forward primer and need not be individually designed to be linked to gene specific forward primers.

In various embodiments, barcodes can be releasably attached to a support structure, such as a bead. Therefore, a single bead with multiple copies of barcodes can be partitioned into an emulsion with a cell lysate, thereby allowing labeling of analytes of the cell lysate with the barcodes of the bead. Example beads include solid beads (e.g., silica beads), polymeric beads, or hydrogel beads (e.g., polyacrylamide, agarose, or alginate beads). Beads can be synthesized using a variety of techniques. For example, using a mix-split technique, beads with many copies of the same, random barcode sequence can be synthesized. This can be accomplished by, for example, creating a plurality of beads including sites on which DNA can be synthesized. The beads can be divided into four collections and each mixed with a buffer that will add a base to it, such as an A, T, G, or C. By dividing the population into four subpopulations, each subpopulation can have one of the bases added to its surface. This reaction can be accomplished in such a way that only a single base is added and no further bases are added. The beads from all four subpopulations can be combined and mixed together, and divided into four populations a second time. In this division step, the beads from the previous four populations may be mixed together randomly. They can then be added to the four different solutions, adding another, random base on the surface of each bead. This process can be repeated to generate sequences on the surface of the bead of a length approximately equal to the number of times that the population is split and mixed. If this was done 10 times, for example, the result would be a population of beads in which each bead has many copies of the same random 10-base sequence synthesized on its surface. The sequence on each bead would be determined by the particular sequence of reactors it ended up in through each mix-split cycle. Additional details of example beads and their synthesis is described in International Application Pub. No. WO2016126871A2, which is hereby incorporated by reference in its entirety.

### Computer Embodiments

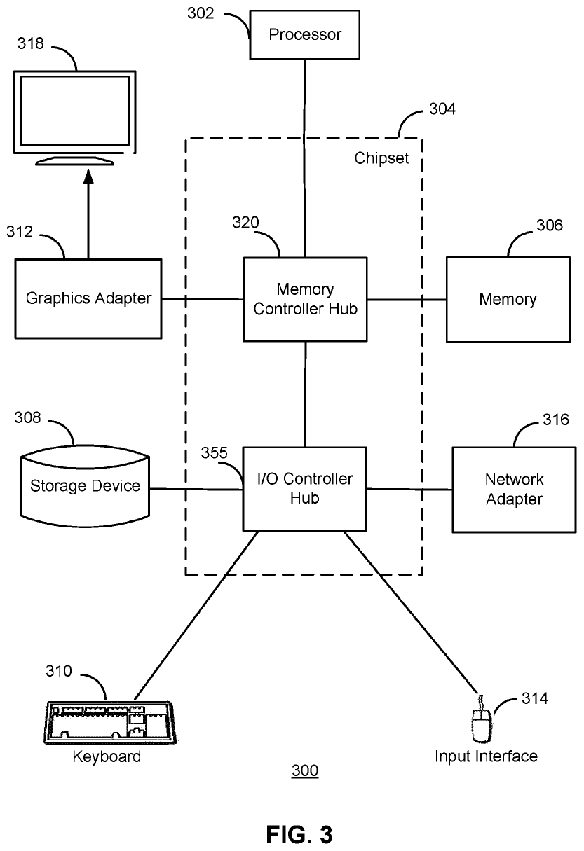

FIG. 3 depicts an example computing device for implementing system and methods described in reference to FIGS. 1A, 1B, 2A, 2B, and 3. In various embodiments, the example computing device 300 serves as the device for performing in silico analysis shown in FIG. 3. Examples of a computing device can include a personal computer, desktop computer, laptop, server computer, a computing node within a cluster, message processors, hand-held devices, multi-processor systems, microprocessor-based or programmable consumer electronics, network PCs, minicomputers, mainframe computers, mobile telephones, PDAs, tablets, pagers, routers, switches, and the like.

As shown in FIG. 3, in some embodiments, the computing device 300 includes at least one processor 302 coupled to a chipset 304. The chipset 304 includes a memory controller hub 320 and an input/output (I/O) controller hub 355. A memory 306 and a graphics adapter 312 are coupled to the memory controller hub 320, and a display 318 is coupled to the graphics adapter 312. A storage device 308, an input interface 314, and network adapter 316 are coupled to the I/O controller hub 355. Other embodiments of the computing device 300 have different architectures.

The storage device 308 is a non-transitory computer-readable storage medium such as a hard drive, compact disk read-only memory (CD-ROM), DVD, or a solid-state memory device. The memory 306 holds instructions and data used by the processor 302. The input interface is a touch interface, examples of which can be a touch-screen interface, a mouse (e.g., pointing device 314), track ball, or other type of input interface, a keyboard (e.g., keyboard 310), or some combination thereof, and is used to input data into the computing device 300. In some embodiments, the computing device 300 may be configured to receive input (e.g., commands) from the input interface via gestures from the user. The graphics adapter 312 displays images and other information on the display 318. The network adapter 316 couples the computing device 300 to one or more computer networks.

The computing device 300 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 308, loaded into the memory 306, and executed by the processor 302.

The types of computing devices 300 can vary from the embodiments described herein. For example, the computing device 300 can lack some of the components described above, such as graphics adapters 312, input interface 314, and displays 318. In some embodiments, a computing device 300 can include a processor 302 for executing instructions stored on a memory 306.

Methods described herein can be implemented in hardware or software, or a combination of both. In one embodiment, a non-transitory machine-readable storage medium, such as one described above, is provided, the medium comprising a data storage material encoded with machine readable data which, when using a machine programmed with instructions for using said data, is capable of executing instructions for calling cells and identifying genetically modified cells, as described herein. Embodiments of the methods described above can be implemented in computer programs executing on programmable computers, comprising a processor, a data storage system (including volatile and non-volatile memory and/or storage elements), a graphics adapter, an input interface, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high-level procedural or object-oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

### Additional Embodiments

Disclosed herein is a method for determining whether one or more cells are genetically transduced, the method comprising: obtaining sequence reads derived from the one or more cells; identifying cell barcodes within the sequence reads that pass a total reads cutoff, defining a plurality of positive amplicon, each positive amplicon having a normalized read value that is greater than a dynamic threshold value; determining that at least one of the one or more cells is a genetically transduced cell according to a presence of at least one positive amplicon derived from the cell as indicated by one of the identified cell barcodes.

In various embodiments, the total reads cutoff is defined by a number of human amplicons in a targeted panel. In various embodiments, the total reads cutoff is defined as a product between the number of human amplicons in the targeted panel and a constant value X. In various embodiments, X is any one of 1, 2, 3, 4, 5, 6, 7, 8, 9, or 10. In various embodiments, X is 8.

In various embodiments, the dynamic threshold value is based on a number of amplicons in a targeted panel. In various embodiments, dynamic threshold value is a product between a constant value Y and a mean of amplicon reads. In various embodiments, Y is any one of 0.1, 0.2, 0.3, 0.4, 0.5, 0.6, 0.7, 0.8, 0.9, or 1. In various embodiments, Y is 0.2.

In various embodiments, determining that at least one of the one or more cells is a genetically transduced cell comprises determining that at least Z % of amplicons with the one of the identified cell barcodes are positive amplicons. In various embodiments, Z % is any one of 10%, 20%, 30%, 40%, 50%, 60%, 70%, 80%, or 90%. In various embodiments, Z % is 80%.

### EXAMPLES

Below are examples of specific embodiments for carrying out the present invention. The examples are offered for illustrative purposes only and are not intended to limit the scope of the present invention in any way. Efforts have been made to ensure accuracy with respect to numbers used (e.g., amounts, temperatures, etc.), but some experimental error and deviation should be allowed for.

### Example 1: Cell Calling Using Human Amplicons, and Excluding Viral Amplicons, Improves Number of Called Cells

Cells were genetically modified via lentiviral particles containing a unique genetic sequence. Cells were processed using a single cell workflow (e.g., Tapestri® single cell DNA platform). Single-cell sequencing was performed on 2-5 replicates for each tested sample. After pre-processing the reads, cells were called using performing amplicons. The workflow for variant calling works robustly with the various possible outcomes of a gene perturbation. The workflow is able to identify variants from single nucleotide polymorphisms (SNPs) to large insertions, deletions and also chromosomal translocation events. Custom modules were used to genotype genomic aberrations. This workflow enables identification and quantification of the effects of gene editing experiments.

Generally, targeted sequencing panels were designed to amplify on-target and putative off-target genome editing sites, and to enable detection of translocation in targeted genes. In particular, a targeted sequencing panel was designed to target 44 regions of interest from which amplicons are generated in the single cell workflow. Of the 44 regions of interest, 22 represent sequences present in the human genome and 22 represent sequences present in the viral genome. Thus, the targeted sequencing panel was designed to target and amplify both human and viral regions of interest. Amplicons were then sequenced to generate sequencing data.

The sequencing data were analyzed to perform cell calling. Briefly, fastq files generated by Illumina Nextseq550 sequencers were processed using the Tapestri single-cell DNA Analysis Pipeline for adapter trimming, barcode extraction and correction, reads alignment, and reads mapping to amplicon insert. scDNA-sequencing resulted in a median of 100× coverage per amplicon per cell (IQR 46x).

Barcodes passing a total reads cutoff defined by the number of human amplicons in the panel times 8 reads were carried on for cell calling. Panel performance was calculated as 0.2 times the mean of all amplicon reads for all qualified barcodes. Working amplicons were those that pass the panel performance threshold. Human amplicon (amplicons 23-44) read completeness in each barcode was used to call cells from all barcodes. It requires each cell barcode to have at least 80% data completeness for the working human amplicons.

Specifically, barcode sequences were identified as called cells using a three-step process using the 22 human amplicons in the targeted sequencing panel. The 22 viral amplicons were purposefully excluded from the cell calling method. The three-step process is as follows:
1) Barcodes that pass a total reads cutoff were selected. The total reads cutoff was defined by the number of human amplicons in the panel (e.g., 22 human amplicons) times 8 reads.
2). The panel performance was calculated as 0.2*the mean of all amplicon reads for all qualified barcodes. With a threshold of 0.2*the mean value, functioning amplicons are identified as those that pass the panel performance threshold.
3). Barcodes that correspond to at least 80% functioning amplicons are used for cell calling. Specifically, these barcodes are attributed to individual cells, which represent the called cells.

Comparatively, the sequencing data were additionally analyzed to perform cell calling using both human amplicons and viral amplicons (e.g., the 44 total regions of interest in the target sequencing panel). The same three-step process was used.

Generally, improved cell calling was achieved when only using human amplicons (e.g., 22 human amplicons in the targeted panel) as compared to using both human amplicons and viral amplicons (e.g., 22 human amplicons and 22 viral amplicons in the targeted panel). The methodologies were performed across six samples to ensure reproducibility. Samples 1-6 had various transduction percentages ranging from 50-90%, with different transgenes (different viral sequences) introduced to cells. In particular, the cell caller that only uses the 22 human amplicons, while excluding the 22 viral amplicons achieved an average of 58% increase in cell number across 28 testing runs. Furthermore, the cell calling process is independent of viral amplicon detection and amplification efficiency.

Altogether, this demonstrates that the cell calling procedure is far more accurate when considering only the human amplicons while excluding the viral amplicons. Considering both human amplicons and viral amplicons led to severe under-detection of cells. This may be due to confounding signals arising from the viral amplicons, including the fact that cells may not be fully transduced (e.g., partial transduction or lower levels of transduction) which would lead to the process inadvertently excluding certain barcodes. Conversely, human amplicon signatures may be more consistent across samples and cells, thereby enabling more accurate and replicable calling of individual cells.

### Example 2: Absolute Read Count Cutoffs Identify Genetically Transduced Cells at Sub-Optimal Rates

Given called cells (as described above in Example 1), each cell was next analyzed to determine whether the cell underwent success genetic modification. In this Example, an absolute read counts cutoff methodology was implemented which used a fixed threshold cutoff for distinguishing between presence or absence of viral amplicons. Specifically, various fixed threshold cutoffs were tested to determine the impact of the selected fixed threshold cutoff and the total percent of transduced cells. Generally, when using an absolute read count cutoff, detection of genetically transduced cells is highly dependent on the selected cutoff.

Given 15 viral amplicons in a testing panel, called cells were each analyzed to determine whether there was a presence of any one of the 15 viral amplicons based on a selected fixed threshold cutoff. Four different fixed threshold cutoffs were tested: 1) 1 read, 2) 5 reads, 3) 10 reads, and 4) 20 reads. Generally, the lower the fixed threshold, the higher the calculated number of transduced cells. Conversely, the higher the fixed threshold, the lower the calculated number of transduced cells. For example, given a fixed threshold cutoff of greater than or equal to 1 read, generally between 60-80% of the cells in the population satisfied the fixed threshold cutoff, thereby indicating that the genetic transduction method achieved between 60-80% successful genetic transduction efficiency. However, given a higher fixed threshold cutoff of greater than or equal to 20 reads, generally between 40-60% of the cells in the population satisfied the fixed threshold cutoff, thereby indicating that the genetic transduction method achieved between 40-60% successful genetic transduction efficiency. This is a drawback of the fixed threshold cutoff methodology as the overall transduction percentage can highly depend on the selected threshold cutoff.

When implementing a fixed threshold of greater than or equal to 1 read, the methodology identified a 94.33 mean transduction percentage, with a coefficient of variation (CV) percentage of 1.118. Here, CV percentage is a variability measure which represents how consistent the mean transduction percentage is across multiple runs. When implementing a fixed threshold of greater than or equal to 5 reads, the methodology identified a much lower 80.54 mean transduction percentage, with a coefficient of variation (CV) percentage of 0.952.

### Example 3: Dynamic Read Normalization Method Identifies Genetically Transduced Cells Accurately and Consistently

In this Example, a dynamic normalization methodology was implemented as opposed to the fixed cutoff methodology described in Example 2.

Generally, the read normalization method using human amplicon reads of each barcode was adapted to remove bias and reduce variation by sequencing depth and number of working amplicons across sequencing runs. For each cell barcode corresponding to a called cell, individual amplicon read count was divided by the total reads of hg19 amplicons to obtain hg19-nr % (hg19 normalized read %). Amplification of 22 TERT and RPPH1 amplicons were used to assess panel performance. The hg19-nr % normalization factor was then applied to all viral amplicons for detection of positive amplification. A transduction positive cell is defined as detection of any positive viral amplicons (any one of amplicons 1-22 in the targeted panel). Dynamic thresholds ranging from 0.05% to 4.5% were evaluated in detection of positive viral amplification and positive transduction using the above method. Coefficient of variation (CV) % of transduction % was calculated (CV %=100× sample standard deviation (STDEV.S)/mean) across duplicates of testing samples to measure assay variability.

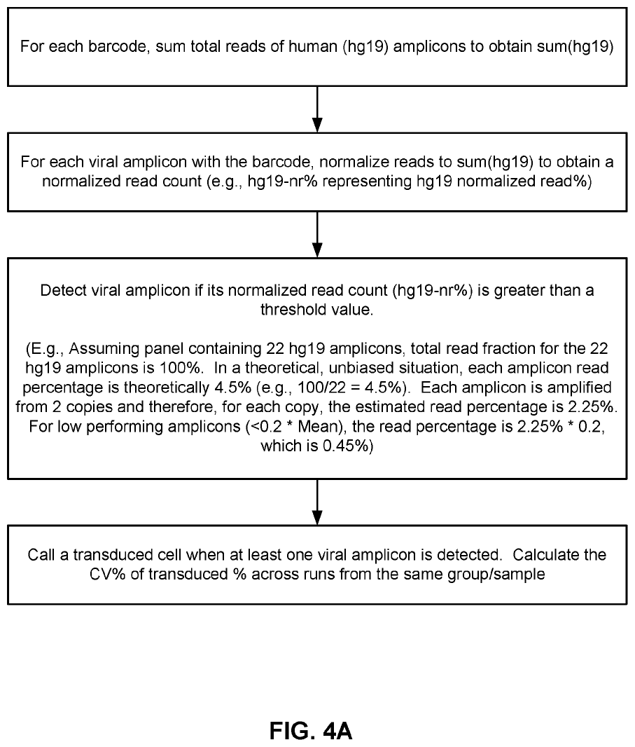

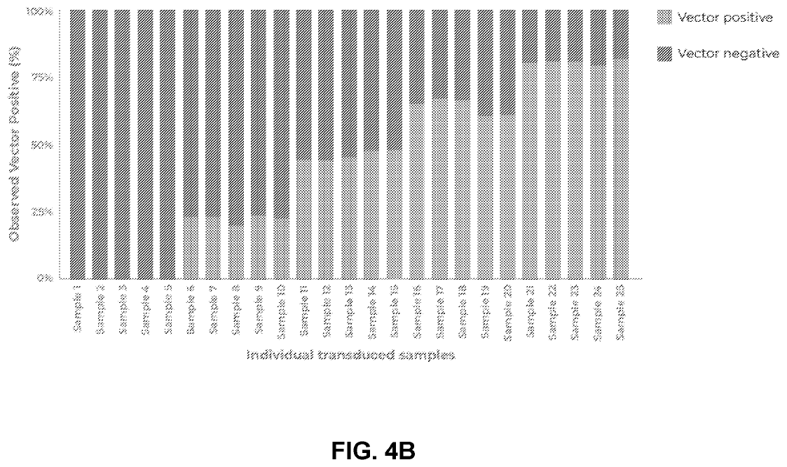

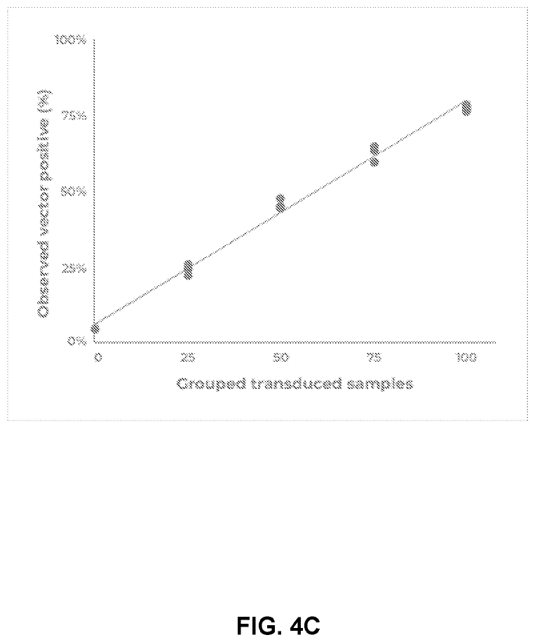

Specifically, FIG. 4A depicts the dynamic normalization method for detecting transduced cells using example hg19 amplicons. Terminology shown in FIG. 4A is further defined below in Equations (1) and (2).

$$hg 19 - nr \% ( hg 19 normalizing read \% ) = ( viral amplicon reads ) / sum ( hg 19 amplicon reads ) \qquad ( 1 )$$

$$Mean = ( sum of amplicon hg 19 - nr \% across all cells ) / ( total cell number ) \qquad ( 2 )$$

Specifically, the dynamic normalization method involved summing the total reads of human amplicons with the barcode. Next, for each viral amplicon with the barcode, a normalized read count for the viral amplicon was generated. Here, the total number of viral amplicons with the barcode were normalized against the total reads of the human amplicons with the barcode. This percentage is denoted as “hg19-nr %.”

This normalize value (e.g., “hg19-nr %”) is compared against a threshold value. In the example shown in FIG. 4A, the threshold value is selected as 0.45%. This threshold value was selected because it represented a theoretical value that would distinguish between low performing amplicons (as denoted as “<0.2*Mean”) and functioning amplicons. Thus, if the normalized value (e.g., “hg19-nr %”) for a viral amplicon was greater than the threshold value, then the viral amplicon was deemed to be present.

The presence of one viral amplicon was sufficient to call a cell as successfully transduced. Using this methodology, the total number of successfully transduced cells in the population of cells was determined.

Generally, the dynamic read normalization method successfully identified genetically transduced cells with an improved coefficient of variation (CV) in comparison to the absolute read count thresholding method (e.g., described in Example 2). The dynamic normalization method considers variation of sequencing depth and number of working amplicons of each run within comparison groups.

When implementing the absolute read count thresholding method using a threshold of greater than or equal to 5 reads, the mean transduction percentage was 80.54% with a CV % of 0.952. The nontransduced percentage was determined to be 0.01%. In contrast, using a threshold of 0.45%, the mean transduction percentage was 80.295% with a coefficient of variation (CV) percentage of 0.865. The nontransduced percentage was determined to be 0.01%. Importantly, in comparison to the results of the absolute read cutoff shown, the CV percentage of the dynamic normalization method (CV % of 0.865) was lower than the absolute read cutoff scenario (e.g., 5 read threshold achieved a CV % of 0.952). This indicates that the results of the dynamic normalization method were more reproducible across multiple runs in comparison to the absolute read cutoff scenario which resulted in more variable results across multiple runs.

Additional comparisons were conducted on a mix of genetically transduced and non-genetically transduced cells. Specifically, comparisons were conducted for a mixture of cells in which 50% of cells were genetically transduced and 50% of cells were not genetically transduced (referred to as 50:50 mixture), a mixture of cells in which 75% of cells were genetically transduced and 25% of cells were not genetically transduced (referred to as 75:25 mixture), and a mixture of cells in which 25% of cells were genetically transduced and 75% of cells were not genetically transduced (referred to as 25:75 mixture). Using the absolute read count threshold method with a 5 read threshold to analyze the 50:50 mixture, it detected a mean transduction percentage of 46.15% with a CV of 3.682%. Using the absolute read count threshold method with a 5 read threshold to analyze the 75:25 mixture, it detected a mean transduction percentage of 64.17% with a CV of 4.193%. Using the absolute read count threshold method with a 5 read threshold to analyze the 25:75 mixture, it detected a mean transduction percentage of 22.50% with a CV of 6.376%. In contrast, using the dynamic read normalization method to analyze the 50:50 mixture, it detected a mean transduction percentage of 45.119% with a CV of 3.36%. Using the dynamic read normalization method to analyze the 75:25 mixture, it detected a mean transduction percentage of 63.92% with a CV of 3.599%. Using the dynamic read normalization method to analyze the 25:75 mixture, it detected a mean transduction percentage of 21.83% with a CV of 5.891%. In every instance, the dynamic read normalization method achieved a lower CV % in comparison to the absolute read count threshold method.

Additionally, further runs and analyses were conducted to determine the impact on the detected mean transduction percentage as a result of subsampling (e.g., 50% subsampling). Subsampling is a measure of the sequencing depth. Therefore, a 50% subsampling refers to a reduction of the sequencing depth by 50%. This is valuable in situations where reduced reads are present (e.g., in situations involving viral amplicons in which a reduced number of viral reads are obtained).

At a 50% subsampling using the absolute read count threshold of 5 reads, the mean transduction rate dropped from 80.54% to 79.55% while the CV % increased from 0.952 to 1.18. This indicates that if sequencing depth is reduced (e.g., reduced by 50%), the absolute read count threshold method would be negatively impacted. Conversely, at a 50% subsampling using the dynamic read normalization method, the mean transduction percentage remains consistent (e.g., 80.295% versus 80.408%). This indicates that even in situations where there are 50% fewer reads (e.g., reads derived from viral amplicons), the dynamic read normalization method is able to successfully identify transduced cells. Furthermore, the coefficient of variation (CV) percentage at the lower subsamplings remains consistent (e.g., CV % for 50% subsampling is 0.879 which is similar to the CV % of 0.865 without subsampling). Again, this indicates that the dynamic read normalization method remains consistent over multiple runs even in view of reduced sequencing depth.

FIGS. 4B and 4B depict the observed percentages of transduced cells across the five replicates from each concentration. Here, serial dilution experiments showcased the ability to detect and quantify transduced vs. non-transduced cells. The assay demonstrated good reproducibility, low false positive rate (<0.03%), and dilution series thereafter showed excellent linearity and precision among replicates between the expected and observed transduction percentages.

Edits were made via lentiviral particles containing a unique genetic sequence and transduced cells were diluted with non-transduced cells to roughly achieve the following percentages of transduced cells: 0, 25, 50, 75, and 100%. Five replicates from each concentration of transduced cells were quantified using the Tapestri® Platform and analyzed with Tapestri® Pipeline and Tapestri® Insights software.

Single-cell sequencing using the Tapestri® Platform was performed on 5 replicates from each concentration of transduced cells. FIGS. 4B and 4B depict the observed percentages of transduced cells across the five replicates from each concentration. Single-cell sequencing across 25 samples in a serial dilution experiment showcased the ability to detect and quantify transduced versus non-transduced cells at expected ratios. For the 5 samples made up of non-transduced cells, the false positive rate was below 0.03%. For the 5 samples that made up the starting point, while not at 100% transduced, the dilution series thereafter showed excellent linearity and precision among replicates between the expected and observed transduction percentages. Overall, these data showcase the ability to use a single cell analysis platform (e.g., the Tapestri® Platform) to interrogate thousands of single cells per sample, and garner the percentage of transduced to non-transduced cells in the sample, without the need for any lengthy clonal outgrowth cell culture assays upfront of analysis.

Altogether, single-cell sequencing technology offers exciting new capabilities for the development of in vivo and ex vivo cell and gene therapies. By precisely measuring the presence or absence of DNA integrations from thousands of single cells, researchers can better optimize their protocols and reduce the time to go to market. In this example, when cells were transduced with a viral vector, single-cell analysis showed high correlation between the expected and observed percentages of transduced cells and exceptional precision among sample replicates. In addition, single-cell sequencing shortened the time from manufacturing to testing results from weeks, which is required for clonal outgrowth, down to days. These characteristics of single-cell sequencing streamline both therapy development and release testing of manufactured clinical cell and gene therapy products.

1. A method comprising:
obtaining reads determined using a targeted sequencing panel, the reads sequenced from amplicons derived from a plurality of cells;
calling one or more of the cells, wherein the calling comprises:
identifying one or more cell barcodes in the obtained reads, wherein the one or more cell barcodes are identified at least for satisfying a total reads cutoff that is defined based on a number of amplicons of a first type in the targeted sequencing panel;
attributing one of the identified one or more cell barcodes to one of the one or more cells;
for each of one or more of the called cells,
determining presence or absence of one or more amplicons of a second type comprising a barcode attributed to the called cell;
determining whether the called cell is a genetically modified cell according to the presence or absence of one or more amplicons of the second type comprising the barcode attributed to the called cell.

2. The method of claim 1, wherein the plurality of cells are a plurality of human cells, and wherein the amplicons of the first type comprise human amplicons.

3. The method of claim 1 or 2, wherein the total reads cutoff is not defined based on a number of amplicons of the second type in the targeted sequencing panel.

4. The method of any one of claims 1-3, wherein the amplicon of the second type is from a source that is foreign to the one or more cells.

5. The method of any one of claims 1-4, wherein the amplicon of the second type is a non-human amplicon.

6. The method of any one of claims 1-5, wherein the amplicon of the second type is a viral amplicon.

7. The method of claim 6, wherein the viral amplicon is derived from a lentivirus.

8. The method of any one of claims 1-7, further comprising:
after obtaining reads determined using a targeted sequencing panel, distinguishing between reads suspected to be sequenced from amplicons of the second type and reads sequenced from amplicons of the first type.

9. The method of any one of claims 1-8, wherein the total reads cutoff is defined as a product between the number of human amplicons in the targeted panel and a constant value X.

10. The method of claim 9, wherein X is any one of 1, 2, 3, 4, 5, 6, 7, 8, 9, or 10.

11. The method of claim 10, wherein X is 8.

12. The method of any one of claims 1-11, wherein the one or more cell barcodes are each identified further based on a performance value of the targeted sequencing panel.

13. The method of claim 12, wherein the performance value of the targeted sequencing panel is a product between a constant value Y and a mean coverage of a subset of reads.

14. The method of claim 13, wherein the subset of reads represent reads comprising cell barcodes that satisfy the total reads cutoff.

15. The method of claim 13, wherein Y is any one of 0.1, 0.2, 0.3, 0.4, 0.5, 0.6, 0.7, 0.8, 0.9, or 1.

16. The method of claim 15, wherein Y is 0.2.

17. The method of any one of claims 1-16, wherein the identified one or more cell barcodes in the obtained sequence reads represent functioning amplicons.

18. The method of any one of 1-17, wherein attributing one of the identified one or more cell barcodes to one of the one or more human cells comprises attributing each identified one or more cell barcodes to a corresponding human cell.

19. The method of any one of claims 1-18, wherein determining presence or absence of one or more amplicons of the second type comprises normalizing reads suspected to be sequenced from amplicons of the second type to reads sequenced from amplicons of the first type.

20. The method of claim 19, wherein the normalization accounts for variations in sequencing depth and quantity of reads across different cells or across different samples.

21. The method of claim 19 or 20, wherein determining presence or absence of one or more amplicons of the second type further comprises comparing the normalized reads suspected to be sequenced from amplicons of the second type to a percentage read cutoff.

22. The method of claim 21, wherein the percentage read cutoff is between 0.05% and 0.5%.

23. The method of claim 21, wherein the percentage read cutoff is any of 0.05%, 0.1%, 0.15%, 0.2%, 0.25%, 0.3%, 0.35%, 0.4%, 0.45%, or 0.5%.

24. The method of any one of claims 1-23, wherein determining whether the called cell is a genetically modified cell according to the presence or absence of one or more amplicons of the second type comprises determining that the called cell is a genetically modified cell when at least one amplicon of the second type is determined to be present.

25. The method of any one of claims 1-24, wherein determining whether the called cell is a genetically modified cell according to the presence or absence of one or more amplicons of the second type comprises determining that the called cell is a genetically modified cell when at least two, at least three, at least four, or at least five amplicons of the second type are determined to be present.

26. The method of any one of claims 1-25, wherein genetically modified cells are successfully identified at a coefficient of variability (CV) percent of less than 1%.

27. The method of any one of claims 1-25, wherein genetically modified cells are successfully identified at a coefficient of variability (CV) percent of less than 0.9%, less than 0.8%, less than 0.7%, less than 0.6%, or less than 0.5%.

28. The method of any one of claims 1-27, wherein the method identifies a consistent mean percentage of genetically modified cells at 100% sampling and at 50% subsampling.

29. The method of any one of claims 1-28, wherein the reads determined using a targeted sequencing panel are obtained by performing a single cell analysis.

30. The method of claim 29, wherein the single cell analysis is one of a single cell DNA analysis or a single cell RNA analysis.

31. The method of any one of claims 1-30, wherein the targeted sequencing panel comprises at least 10 amplicons of the first type and at least 10 amplicons of the second type.

32. The method of any one of claims 1-30, wherein the targeted sequencing panel comprises at least 20 amplicons of the first type and at least 20 amplicons of the second type.

33. A non-transitory computer readable medium comprising instructions that, when executed by a processor, cause the processor to:
obtain reads determined using a targeted sequencing panel, the reads sequenced from amplicons derived from a plurality of cells;
call one or more of the cells, wherein the instructions that cause the processor to call the one or more of the cells further comprises instructions that, when executed by the processor, cause the processor to:
identify one or more cell barcodes in the obtained reads, wherein the one or more cell barcodes are identified at least for satisfying a total reads cutoff that is defined based on a number of amplicons of a first type in the targeted sequencing panel;
attribute one of the identified one or more cell barcodes to one of the one or more cells;
for each of one or more of the called cells,
determine presence or absence of one or more amplicons of a second type comprising a barcode attributed to the called cell;
determine whether the called cell is a genetically modified cell according to the presence or absence of one or more amplicons of the second type comprising the barcode attributed to the called cell.

34. The non-transitory computer readable medium of claim 33, wherein the plurality of cells are a plurality of human cells, and wherein the amplicons of the first type comprise human amplicons.

35. The non-transitory computer readable medium of claim 33 or 34 wherein the total reads cutoff is not defined based on a number of amplicons of the second type in the targeted sequencing panel.

36. The non-transitory computer readable medium of any one of claims 33-35, wherein the amplicon of the second type is from a source that is foreign to the one or more cells.

37. The non-transitory computer readable medium of any one of claims 33-36, wherein the amplicon of the second type is a non-human amplicon.

38. The non-transitory computer readable medium of any one of claims 33-37, wherein the amplicon of the second type is a viral amplicon.

39. The non-transitory computer readable medium of claim 38, wherein the viral amplicon is derived from a lentivirus.

40. The non-transitory computer readable medium of any one of claims 33-39, further comprises instructions that, when executed by the processor, cause the processor to:
after obtaining reads determined using a targeted sequencing panel, distinguish between reads suspected to be sequenced from amplicons of the second type and reads sequenced from amplicons of the first type.

41. The non-transitory computer readable medium of any one of claims 33-40, wherein the total reads cutoff is defined as a product between the number of human amplicons in the targeted panel and a constant value X.

42. The non-transitory computer readable medium of claim 41, wherein X is any one of 1, 2, 3, 4, 5, 6, 7, 8, 9, or 10.

43. The non-transitory computer readable medium of claim 42, wherein X is 8.

44. The non-transitory computer readable medium of any one of claims 33-43, wherein the one or more cell barcodes are each identified further based on a performance value of the targeted sequencing panel.

45. The non-transitory computer readable medium of claim 44, wherein the performance value of the targeted sequencing panel is a product between a constant value Y and a mean coverage of a subset of reads.

46. The non-transitory computer readable medium of claim 45, wherein the subset of reads represent reads comprising cell barcodes that satisfy the total reads cutoff.

47. The non-transitory computer readable medium of claim 45, wherein Y is any one of 0.1, 0.2, 0.3, 0.4, 0.5, 0.6, 0.7, 0.8, 0.9, or 1.

48. The non-transitory computer readable medium of claim 47, wherein Y is 0.2.

49. The non-transitory computer readable medium of any one of claims 33-48, wherein the identified one or more cell barcodes in the obtained sequence reads represent functioning amplicons.

50. The non-transitory computer readable medium of any one of 33-49, wherein the instructions that cause the processor to attribute one of the identified one or more cell barcodes to one of the one or more human cells further comprises instructions that, when executed by the processor, cause the processor to attribute each identified one or more cell barcodes to a corresponding human cell.

51. The non-transitory computer readable medium of any one of claims 33-50, wherein the instructions that cause the processor to determine presence or absence of one or more amplicons of the second type further comprises instructions that, when executed by the processor, cause the processor to normalize reads suspected to be sequenced from amplicons of the second type to reads sequenced from amplicons of the first type.

52. The non-transitory computer readable medium of claim 51, wherein the normalization accounts for variations in sequencing depth and quantity of reads across different cells or across different samples.

53. The non-transitory computer readable medium of claim 51 or 52, wherein the instructions that cause the processor to determine presence or absence of one or more amplicons of the second type further comprises instructions that, when executed by the processor, cause the processor to compare the normalized reads suspected to be sequenced from amplicons of the second type to a percentage read cutoff.

54. The non-transitory computer readable medium of claim 53, wherein the percentage read cutoff is between 0.05% and 0.5%.

55. The non-transitory computer readable medium of claim 54, wherein the percentage read cutoff is any of 0.05%, 0.1%, 0.15%, 0.2%, 0.25%, 0.3%, 0.35%, 0.4%, 0.45%, or 0.5%.

56. The non-transitory computer readable medium of any one of claims 33-55, wherein the instructions that cause the processor to determine whether the called cell is a genetically modified cell according to the presence or absence of one or more amplicons of the second type further comprises instructions that, when executed by the processor, cause the processor to determine that the called cell is a genetically modified cell when at least one amplicon of the second type is determined to be present.

57. The non-transitory computer readable medium of any one of claims 33-56, wherein the instructions that cause the processor to determine whether the called cell is a genetically modified cell according to the presence or absence of one or more amplicons of the second type further comprises instructions that, when executed by the processor, cause the processor to determine that the called cell is a genetically modified cell when at least two, at least three, at least four, or at least five amplicons of the second type are determined to be present.

58. The non-transitory computer readable medium of any one of claims 33-57, wherein genetically modified cells are successfully identified at a coefficient of variability (CV) percent of less than 1%.

59. The non-transitory computer readable medium of any one of claims 33-58, wherein genetically modified cells are successfully identified at a coefficient of variability (CV) percent of less than 0.9%, less than 0.8%, less than 0.7%, less than 0.6%, or less than 0.5%.

60. The non-transitory computer readable medium of any one of claims 33-59, wherein the method identifies a consistent mean percentage of genetically modified cells at 100% sampling and at 50% subsampling.

61. The non-transitory computer readable medium of any one of claims 33-60, wherein the reads determined using a targeted sequencing panel are obtained by performing a single cell analysis.

62. The non-transitory computer readable medium of claim 61, wherein the single cell analysis is one of a single cell DNA analysis or a single cell RNA analysis.

63. The non-transitory computer readable medium of any one of claims 33-62, wherein the targeted sequencing panel comprises at least 10 amplicons of the first type and at least 10 amplicons of the second type.

64. The non-transitory computer readable medium of any one of claims 33-62, wherein the targeted sequencing panel comprises at least 20 amplicons of the first type and at least 20 amplicons of the second type.
